(19) United States
(12) Patent Application Publication
(10) Pub. No.: US 2010/0056964 A1
(43) Pub. Date: Mar. 4, 2010

(54) Backrest Device

(75) Inventor: Neil Summers, Surrey (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ENANEF Limited, Dorking (GB)

(21) Appl. No.: 12/548,231

(22) Filed: Aug. 26, 2009

(30) Foreign Application Priority Data
Sep. 1, 2008 (GB) 0815863.6

(51) Int. Cl. A61H 23/00; A61F 7/00; A47C 16/00

(52) U.S. Cl. 601/49; 607/108

(57) ABSTRACT

The invention provides for a backrest device having an upwardly facing convex form defined generally by upper convex surface, and which device is provided at remote ends of the undersurface thereof with feet for the floor-mounting of the device. Engagement formations are provided extending upwardly from the upper convex surface and, in a particular embodiment, each provides for a combination heating/cooling element which can be readily controlled so as to apply a healing and/or cooling effect to the musculature surrounding the human spine, the bony parts of which are readily located within a recess channel extending centrally in the longitudinal direction of the device and between respective rows of engagement formations.

### BACKGROUND OF THE INVENTION

The present invention relates to a backrest device and in particular to a device allowing for the stretching of the user's spine when lying thereon.

A wide variety of devices are known for the manipulation and massaging of the human back and in particular the spine and related musculature, and which can be used in relation to the human back when in a variety of positions.

One known form of backrest device for use by a user lying thereon is known from EP-A-0 741 533.

The subject matter of which relates to a backrest device having a convex curvature that provides for stretching of the spine, and which is further provided with so-called knobbles arranged to apply pressure to the musculature either side of the spine.

The subject matter of EP-A-0 741 533 is however very much limited to the tactile mechanical application of a stretching/massaging force, and the present invention seeks to provide for a backrest device offering further improvements and advantages over known such devices.

### SUMMARY OF THE INVENTION

According to the present invention there is provided a backrest device arranged to engage the back of a user when lying thereon and having an upwardly facing convex form, the device further including temperature control means for controlling the temperature of at least a portion of the device for application of a heating and/or cooling effect to the user's back.

The present invention can prove advantageous insofar as at the time of providing for the all important stretching of the human back, a heating and/or cooling effect can be applied as required which could be in a general, or localised, manner so as to enhance the range of treatments, and possible advantageous effects of using the backrest device. In one particular embodiment, the backrest device can be arranged with a resilient support member extending from the underside thereof and which serves to support the device on a floor surface in a resilient manner.

Alternatively, or in addition, the backrest device can include a support member extending from an underside thereof and which is adjustable so as to adjust the height of one end of the device with respect to the other.

Of course, advantageously, the said support means for selectively varying the height of one end of the backrest device can also comprise the aforementioned resilient support member.

When in use, at least one end of the backrest device can then reciprocate in a resilient manner and in response to the movement of the user, which we might of course be undertaken as part of an exercise routine.

With regard to the temperature control elements, these can be provided integral with the backrest device or in a manner with to be removable, and replaced therefrom.

The means for providing a cooling effect can, in particular, comprise any appropriate form of cooling-pack/mat, which can be removed and replaced as required with regard to the desired cooling effect.

Any such pack/mat can have a configuration determined by the spacing and configuration of any heating and/or vibratory means such that the cooling pack/mat can extend adjacent thereto.

It will be appreciated that any appropriate form of cooling pack/mat can be provided whether an endothermic chemical ice pack/mat or otherwise.

In one arrangement, the backrest device includes at least one electrically driven heating element.

Alternatively, or in addition, the backrest device can include at least one electrically driven cooling element.

The control means can then advantageously be provided for the predetermined control of a heating and/or cooling pattern wherein one of at least the application time, or maximum temperature, can be readily controlled in a predetermined manner if required.

In particular, the control means can be arranged for providing a cyclical healing/cooling pattern by way of the backrest device.

Further, the backrest device can be provided with upstanding engagement formations along the length thereof and arranged for engaging with a user's back on either side of the spine.

Preferably, two substantially parallel rows of upstanding engagement formations are provided for engaging with the musculature on either side of the spine.

Preferably, at least one of the upstanding engagement formations comprises a heating element the temperature of which is arranged to be controlled by the said control means.

Alternatively, or in addition, at least one of the upstanding engagement formations includes a cooling element the temperature of which is arranged to be controlled by the said control means.

Yet further, at least one of the engagement formations can comprise a combination heating/cooling element which can prove particularly beneficial when the control means is arranged to provide for the aforesaid cyclical temperature change pattern.

Preferably, each upstanding engagement formation is dome-like and, preferably, substantially circular in configuration.

this manner, the circular engagement formation can be provided with a central circular element which provides for one of heating or cooling, and an outer annular portion which provides for the other of heating or cooling.

Preferably, the surface of the engagement formation comprises a plurality of secondary engagement formations. As such each engagement formation can have a dimpled surface.

Preferably, when arranged in rows, the dimensions of the engagement formations differ in the longitudinal direction of the device.

Preferably, the engagement formations increase in lateral dimension and, in particular. in diameter in the longitudinal direction of the device.

Yet further, the backrest device includes a vibratory unit arranged to cause the device to vibrate and thus impart vibration to a user lying thereon.

Further, control means can be provided for the vibratory unit so as to allow for a predetermined cyclical pattern of vibration.

In one particular embodiment the convex curvature of the said convex form increases towards one end.

It should be appreciated that the present invention can advantageously therefore provide for not only treatment, whether therapeutic or otherwise, of a user's back, but also exercising of a user's body and, in particular, the abdominal muscles thereof.

Indeed, the present invention can provide for a backrest device arranged to engage the back of the user when lying thereon and having an upwardly facing convex form, with one end exhibiting a greater degree of convex curvature than the other, and the device further including resilient support means for supporting the device on a floor surface.

Additionally, or alternatively, the backrest device can be provided with support means arrange to extend from an underside thereof, and also arrange to vary the height of one end of the device relative to the other.

Such a device can then be focussed particularly upon exercise routines that can be offered to a user.

### BRIEF DESCRIPTION OF THE DRAWINGS

The invention is described further hereinafter, by way of example only, with reference to the accompanying drawings in which:

FIG. 1 is a perspective view of the backrest device according to an embodiment of the present invention; and

FIG. 2 is a perspective end view of the device of FIG. 1;

FIG. 3 is a view from the said one end of the device of FIG. 1;

FIG. 4 is a perspective view of a backrest device according to another embodiment of the present invention;

FIG. 5 is a side elevational view of the embodiment illustrated in FIG. 4.

### DETAILED DESCRIPTION OF THE INVENTION

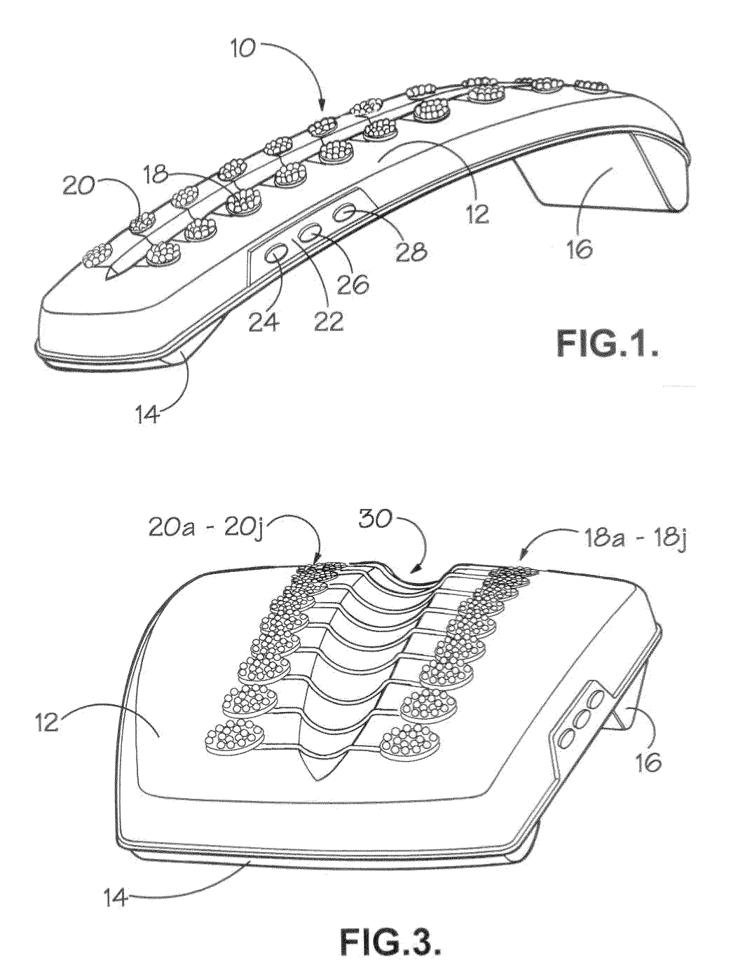

Turning first to FIG. 1, there is illustrated in perspective view a backrest device which is arranged to engage the back of a user when lying thereon and which, as is described further below, is arranged to provide for beneficial massaging/stretching of the human back in a readily controlled and further enhanced manner.

The backrest device 10 offers a generally upwardly facing convex form defined in particular by an upper surface 12 opposite which there is an under-surface presenting at either end respective feet 14, 16 arranged for secure and stable location of the device when in use.

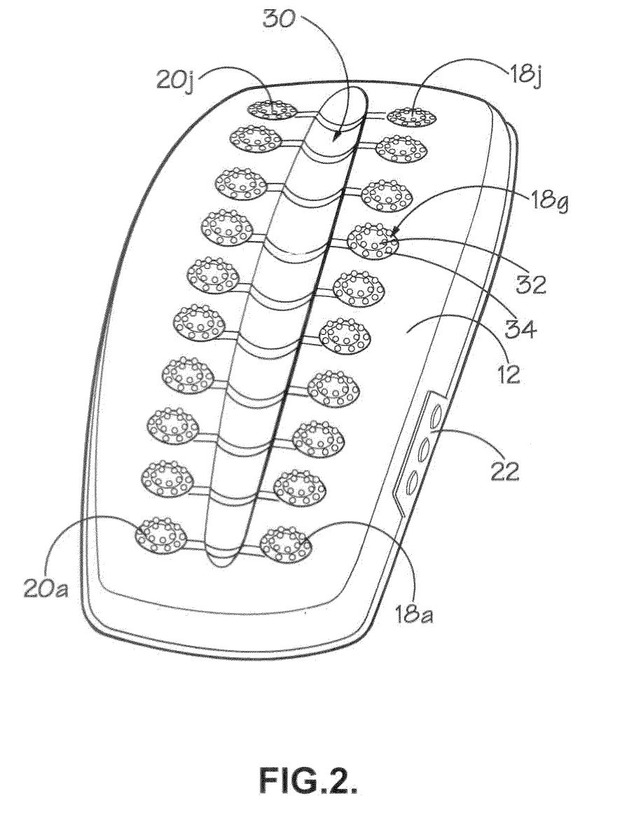

Extending from the upper surface 12 of the device 10 are a series of upstanding dome-like engagement formations 18, 20 which are provided in respective rows as will be described further below with reference in particular, to FIG. 2.

As will also be further described, the dome-like formations 18, 20 are arranged to engage with the musculature either side of the human spine with the user lying on the device.

A particular feature of the illustrated embodiment of the present invention is that the temperature of each formation 18, 20 can be readily controlled so as to provide a warming/heating effect and/or a cooling effect to the user. Likewise, an electrically-driven vibratory unit is mounted within the device 10 such that, when activated, the device can be arranged to vibrate, with such vibrations transmitted to the user by way of the upstanding dome-like formations 18, 20.

A control panel 22 is provided at the side of the device 10 and includes a series of press buttons 24-28 from which the application of heating effect/cooling effect and/or vibratory motion and any combinations thereof, can be provided in a readily controlled, predetermined and/or programmable, manner.

Turning now to FIG. 2, there is illustrated in greater detail the upper surface 12 of the device 10 and in which can clearly be seen two rows 18a-18j; 20a-20j of dome-like engagement formations 18,20.

As will be appreciated, these two rows are substantially parallel and separated by an elongate channel 30 extending longitudinally therebetween.

The channel 30 is provided so as to increase the ease and comfort within which the user's back is received thereon and the device can be used.

As is also illustrated in FIG. 2, the dimensions of the dome-like engagement formations can vary in the longitudinal direction of the device that is, in the direction from the engagement formation 18a to engagement formation 18g, the engagement formations increase in size and, in particular, in diameter.

Referring particularly to dome-like engagement formation 18g, the concentric configuration of the heating 32 and cooling 34 elements thereof can be clearly seen.

That is, a central circular portion comprises a heating element 32, and an outer annular region 34 comprises a cooling element.

From the above, it should be appreciated that in the illustrated embodiment, each of the dome like engagement formations 18, 20 comprises a combinational heating/cooling element. Of course, as an alternative any one or more of the dome-like engagement formations 18, 20 can comprise solely a healing element or solely a cooling element.

With reference to FIG. 3, the form of the backrest device of the present invention can be further appreciated and, in particular, the depth of the channel 30 provided for accommodating the user's spine. The surfaces of each of the engagement formations 18,20 can therefore be provided in a dimpled, or otherwise varied, manner, so as to provide an appropriate degree of texture to assist with the manipulation/manage offered by the device.

Also, the increase in diameter of the engagement formations 18, 20 is likewise clearly-visible in FIG. 3.

In use therefore, and with regard to FIG. 1, the user finds a comfortable position with their back stretching over the upwardly facing convex form of the device 10, and with head and shoulders located towards the end of the device 10 offering the greater curvature.

In this manner, the user's back is not only subject to a beneficial stretching force but each of the upstanding engagement formations 18, 20 serves to engage with, and press into, the musculature associated with the spine.

With the spine stretched, and the musculature engaged in this manner, the temperature control means of the device 10 can be readily operated by appropriate manipulation of the buttons 24-28 of the control panel 22 such that localised heating and/or cooling can be provided by way of the temperature controlled engagement formations 18, 20.

The amount of heating/cooling applied, and/or the time for which such heating/cooling is applied, can be readily controlled in manual manner or, as required, in any predetermined/programmed and appropriate manner insofar as the control means advantageously provides for such possibilities.

In particular, the control means can be arranged to provide for a cyclical heating/cooling pattern whereby each of the upstanding engagement formations 18, 20 cycles through a heating, and then a cooling, phase cycle through repeated heating and cooling phases.

The provision of such localised heating/cooling to the human back by way of the illustrated embodiment of the present invention which back is also undergoing stretching, provides for particular advantageous features of the present invention.

To further enhance the massaging/manipulation offered by the illustrated embodiment of the present invention, a vibratory unit can be employed and activated so as to cause the device, and in particular the upstanding engagement formations 18, 20, to vibrate whilst in engagement with the user's back.

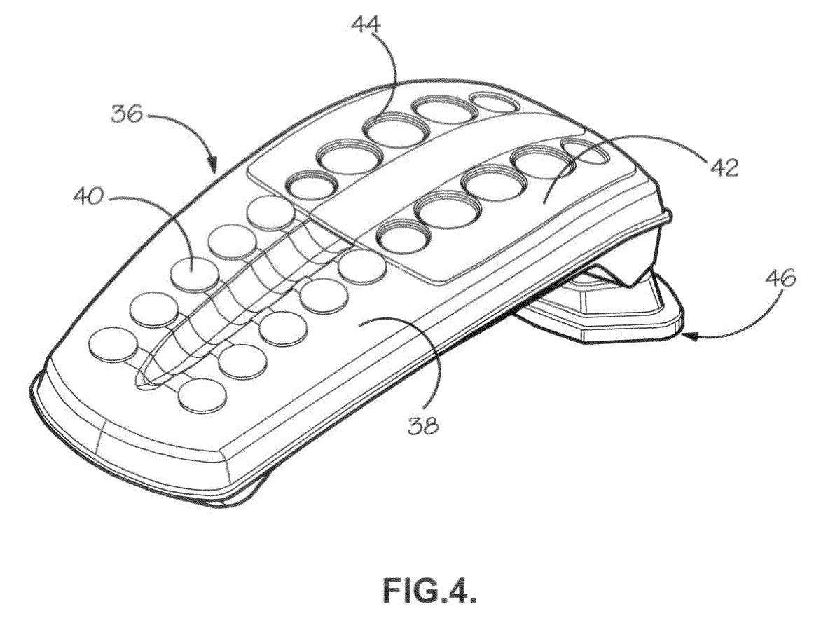

Turning now to FIG. 4, there is provided a perspective view of another embodiment of a backrest device according to the present invention.

The backrest device 36 illustrated in FIG. 4 is a similar overall configuration to that of the proceeding embodiment and again offers an upwardly convex surface 38 over-which a users back is intended to stretch during a treatment and/or exercise regime.

A series of circular formations 40 are again provided in substantially parallel rows and which, as required, can be arranged to provide for a vibratory and/or heating effect or treatment, massage or otherwise for a user's back.

However, for this further embodiment of the present invention, the potential cooling effect is provided by a cooling mat 42 which extends over approximately one half of the upper concave surface 38 of the backrest device 36 and which has two parallel rows of circular apertures 44 provided therein so as to allow for exposure of the formations 40 provided in the region of the cooling mat 42.

Also provided in this further embodiment of the present invention is a selectively adjustable support member 46 arranged to engage with a floor surface upon which the backrest device 36 is located and which is also arranged to provide resilient support for the backrest device 36 as described further hereinafter.

With regard to the cooling mat 42, this can be advantageously provided as a integral member of the backrest device and so as to offer a desired cooling effect and can comprise an electrically driven cooling mat or indeed a simple endothermic cooling mat such as an ice pack mat. Alternatively, the cooling mat 42 can be provided as a separate removable, replaceable member located on the upper convex surface 38 of the backrest 36 as and when required and which, in such an arrangement, can also advantageously comprise an endothermic pack/mat such as a standard icepack/mat.

The required cooling effect can then be provided without requiring electrical input.

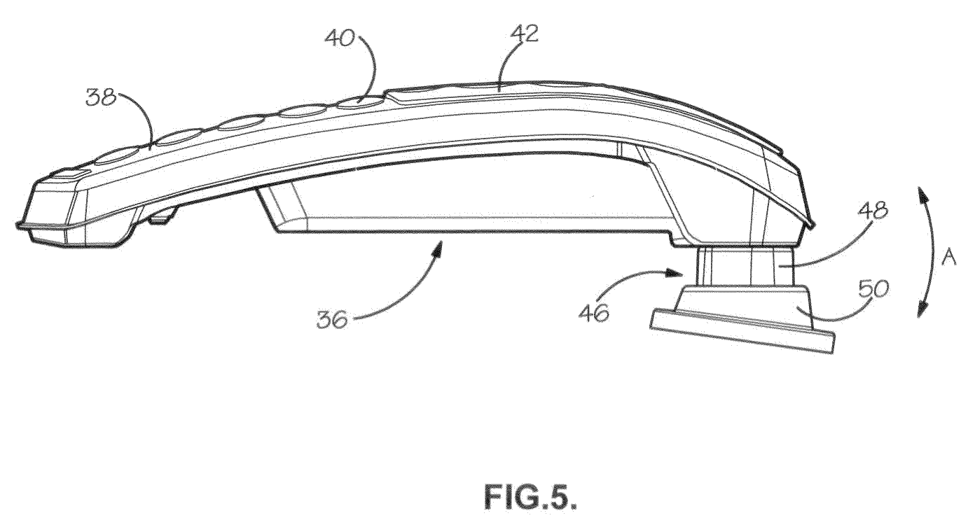

Turning now to FIG. 5, there is provided a side elevational view of the further embodiment of the present invention illustrated with reference to FIG. 4.

Similar reference numbers are again used for the same features and the provision of the cooling mat 42 over some of the series of substantially circular vibratory/heating formations 40 is further illustrated.

FIG. 5 however shows in greater detail the support member 46 which comprises a cylindrical leg 48 extending downwardly into a fool plate 50.

A spring element (not shown) cooperatively engages the underside of the leg 48 and an inner surface of the foot plate 50 so as to allow for resilient movement between the leg 48 and footplate 50 in the manner illustrated by arrow A.

Insofar as the leg 48 can be rigidly connected to the underside of the backrest device 36 it therefore proves readily possible to provide for resilient motion to one end of the backrest device 36.

According to a further feature, a the extent to which the leg 48 extends downwardly from the underside of the backrest device 36 can be selectively controlled and adjusted so as to effectively vary the height of the said one end of the backrest device relative to the other. As a particular advantage, such adjustability in height is provided at the end of the backrest device 36 providing the greater degree of convex curvature.

Such selective adjustment of the relative heights of the two ends of the device, and the resilience offered by the spring coupling between the leg 48 and foot plate 50, serves to enhance the manner in which the backrest device can be used as pan of an exercise routine. In particular, the resilience offered by the resilient coupling between the leg 48 and the foot plate 50 of the support member 46 serve to emphasise the work required to be undertaken by the user lying with their back on the backrest device and while performing an exercise routine and in particular an exercise routine exercising the abdominal muscles.

Yet further, the change in relative height between the respective ends of the device 36 as offered by the selectively adjustable support member 46 can likewise serve to vary the nature of the work undertaken by the user during an exercise routine.

As will therefore be appreciated, the illustrated embodiment of the present invention provides for a particularly adaptable and easily used, backrest device offering either alone, in combination, a variety of treatment scenarios for massaging/manipulating the human as required.

Also, from the above, it will be appreciated that the backrest device can also form an essential part of an exercise device so as to advantageously offer a user muscle strengthening/toning in addition to therapeutic, or otherwise, treatment and manipulation.

It should of course be appreciated that the present invention is not restricted to the details of the foregoing embodiment.

Although the illustrated embodiment is provided in moulded plastic form, it should be appreciated that the device can be made from any appropriate material and can have any appropriate, upper and/or lower, configuration.

The heating and cooling effects can be achieved by any appropriate arrangement, for example through electrical healing wires and at least one cyclic refrigeration unit, but the invention is in no way limited thereto.

Also, the application of the heating/cooling effect need not be provided by way of the upwardly standing engagement formations as illustrated and could, for example, but provided by way of healing elements quite separate from the upstanding engagement formations if indeed present.

What is claimed is:

1. A backrest device arranged to engage the back of a user when lying thereon and having an upwardly facing convex form, the device further including temperature control means for controlling the temperature of at least a portion of the device for application of a healing effect and/or a cooling effect to the back of the user.

2. A device as claimed in claim 1, and including a resilient support means extending from an underside thereof.

3. A device as claimed in claim 1, and including a support member arranged to extend from an underside thereof and arranged for varying the height of a first end of the device relative to a second end of the device.

4. A device as claimed in claim 3, and wherein the said first end of the device presents an upwardly facing convex form having a greater degree of convex curvature than the said second end.

5. A device as claimed in claim 3, wherein the said support member comprises an extendable foot member.

6. A device as claimed in claim 3, wherein the said support member comprises the resilient support means.

7. A device as claimed in claim 1, wherein the said temperature control means is integral with the device.

8. A device as claimed claim 1, wherein at least part of the temperature control means comprises a separate element.

9. A device as claimed in claim 8, wherein the said separate element comprises a replaceable element.

10. A device as claimed in claim 8, wherein the said separate element comprises means for application of the cooling effect.

11. A device as claimed in claim 1 wherein means for application of the cooling effect comprises at least one cooling mat member.

12. A device as claimed in claim 11, wherein the said cooling mat member is configured to extend adjacent means of providing the heating effect.

13. A device as claimed in claim 11 wherein the cooling mat comprises an ice pack.

14. A device as claimed in claim 11, wherein the cooling mat member comprises an endothermic means.

15. A device as claimed in claim 1, and including at least one electrically driven cooling element.

16. A device as claimed in claim 1 and including at least one electrically driven heating element.

17. A device as claimed in claim 1, wherein the control means is arranged for the predetermined control of a temperature change pattern wherein one of at least the time period of operation or maximum temperature can be readily controlled.

18. A device as claimed in claim 17, wherein the control is arranged for providing a cyclical healing/cooling pattern.

19. A device as claimed in claim 1, and including upstanding engagement formations substantially along the length thereof and arranged for engaging with a user's back on either side of the spine.

20. A device as claimed in claim 19, wherein at least one of the upstanding engagement formations comprises a heating element the temperature of which is arranged to be controlled by the said control means.

21. A device as claimed in claim 19, wherein at least one of the upstanding engagement formations includes a cooling element the temperature of which is arranged to be controlled by the said control means.

22. A device as claimed in claim 19, wherein at least one of the engagement formations comprises a combination heating/cooling element.

23. A device as claimed in claim 1, wherein each upstanding engagement formation has a domed configuration.

24. A device as claimed in claim 1 and having a central portion arranged to provide for one of heating or cooling, and an outer portion arranged to provide for the other of heating or cooling.

25. A device as claimed in claim 1 wherein the surface of at least one engagement formation comprises a plurality of secondary engagement formations.

26. A device as claimed in claim 25, wherein the said one engagement formation can have a dimpled surface.

27. A device as claimed in claim 1 wherein the said engagement formations differ in the longitudinal direction of the device.

28. A device as claimed in claim 1 and including vibratory means.

29. A device as claimed in claim 28 and including control means for the vibratory means and arranged so as to allow for a predetermined cyclical pattern of vibration.
